(19) United States
(12) Patent Application Publication
(10) Pub. No.: US 2021/0186788 A1
(43) Pub. Date: Jun. 24, 2021

(54) DEVICE FOR TRANSCUTANEOUS APPLICATION OF CARBON DIOXIDE AND A FUNCTIONING METHOD OF THE SAID DEVICE

(71) Applicant: Derma art d.o.o., Brezice (SI)

(72) Inventors: Maja TRUDEN, Kranj (SI); Daniel VEZOJA, Domzale (SI)

(21) Appl. No.: 17/115,356

(22) Filed: Dec. 8, 2020

(30) Foreign Application Priority Data
Dec. 24, 2019 (EP) 19219593.1

(51) Int. Cl. A61G 10/02; A61M 16/00; A61M 16/20

(52) U.S. Cl. CPC A61G 10/023; A61M 2202/0225; A61M 16/208; A61M 16/0057

(57) ABSTRACT

The present invention is a device for transcutaneous application of CO₂, which comprises:
a therapeutic chamber comprising at least a portion to receive a part of a patient's body to which the carbon dioxide is to be applied,
an inlet/outlet pipe connecting the chamber with a CO₂ distribution system, the said pipe connecting the chamber with a CO₂ distribution system, wherein a gate valve for sealing the chamber is installed in the chamber where the inlet/outlet pipe is installed;
the CO₂ distribution system comprising a housing where at least the following components are installed:
   a first pipe with a first valve for suction of air out of the chamber,
   a second pipe with a second valve for supplying CO₂ from a tank; wherein the first and second pipe are combined into the inlet/outlet pipe upstream of the valves;
   at least one ventilator for ensuring air flow through the said valves and inlet/outlet pipe,
   an outlet pipe for leading the used air from the chamber through the wall to the external environment of the building where the device is installed; and
at least one tank for storing CO₂ suitably connected to the second pipe.

### FIELD OF THE INVENTION

The present invention belongs to the field of medical devices for treatment with carbon dioxide, more precisely to the field of devices for transcutaneous application of carbon dioxide. The invention also belongs to the field of piping, connections and valves for medical devices and medical use. The invention relates to a device for transcutaneous application of carbon dioxide for treatment of chronic wounds and/or neuropathy, as well as to a functioning method of the said device.

### BACKGROUND OF THE INVENTION

CO₂ can be applied to treat a variety of disorders, mostly treatment for peripheral vascular disorders (Dogliotti et al, 2011, Int Angiol. 30(1):12-7.). The benefits of bathing in CO₂-enriched water have been described (Hartmann et al, 1997, https://doi.org/10.1177/000331979704800406; Toriyama et al., 2002, Int Angiol. 21(4):367-73). It has been observed that CO₂ stimulates blood flow and microcirculation to increase partial O₂ pressure in local tissue, which is known as the Bohr effect (Irie et al, 2005, Circulation 111:1523-1529; Bohr et al., 1904, https://doi.org/10.1111/j.1748-1716.1904.tb01382.x). The Bohr effect indeed occurs in human body after transcutaneous administration of CO₂ as shown by Sakai et al. (2011, PloS One, 6, 9: e24137). More recently, CO₂ therapy has been found to induce mitochondrial apoptosis in human tumours, hence it has also been suggested for clinical testing for treatment of primary tumours (Oe et al, 2011, Biochem Biophys Res Commun 407: 148-152; Onishi et al, 2012, https://doi.org/10.1371/journal.pone.0049189; Takeda et al, 2014, PLoS One 9: e100530; Ueha et al, 2107, https://doi.org/10.3892/or.2017.5591).

Usually, CO₂ therapy is performed by bathing in CO₂ enriched water or by injection, wherein transcutaneous CO₂ application using 100% CO₂ gas is supported by applying CO₂ absorption-enhancing hydrogel (Onishi et al, 2012). Both known methods have disadvantages. CO₂ enriched water is prepared by supplying CO₂ in gaseous form from tanks, however the amount of CO₂ in water is small, due to potential danger of inhaling toxic amounts of CO₂. It has been estimated that the concentration of CO₂ in the enriched water is only 0.1% and there is no evidence showing absorption in human body (Hashimoto et al, 2004, J Appl Physiol 96:226-232; Yamamoto et al, 2007, Int J Biometeorol 51:201-208). Further, such bathing is not suitable for hospital treatments and for patients with chronic or acute wounds.

A chronic wound is a wound that does not heal in a usual manner and it is widely accepted that wounds that do not heal within three months are considered chronic. Chronic wounds have different causes (ischemic, neuropathic, etc.) and may differ in the stage of healing in which they are detained. In some cases, such wounds may never heal or take years to do so. Delayed wound healing has also been linked to peripheral neuropathy, which is a condition where peripheral nerves are damaged and cause various unpleasant sensations, including pain. Peripheral neuropathy can be a result of several different causes—traumatic injuries, infections, metabolic problems, inherited causes and exposure to toxins, however one of the most common causes is diabetes. In patients with this metabolic condition, nerve damage tends to lead to a loss of sensation in limbs, usually feet. Wounds on the lower extremities are often overlooked, resulting in delayed wound care and untreated infection that, if it turns gangrenous, may need to be amputated to stop the spread.

Amputations could be decreased, limited or even prevented by improving blood flow and circulation in affected areas. As carbon dioxide has been known to exert these desired effects, there is a need for a device for transcutaneous application of carbon dioxide, which will allow safe treatment of neuropathy or chronic wounds without the need for bathing, injection or any supplemental hydrogel or water-based CO₂ carriers, which could deteriorate the state of wounds in diabetic patients. Thus, the technical problem solved by the present invention is construction of a device and all its parts, which will enable safe delivery of adequate amounts of CO₂ through the patient's skin for treatment.

Several different devices for trans- or subcutaneous delivery of CO₂ into human body are already known, but they use different approaches than the present invention. Mostly, the known solutions are for injection-based therapy (subcutaneous delivery). One such solution is disclosed at web page http://www.mbemedicale.it/en/prodotto/venusian-co2-therapy. This device is primarily intended for use in gynaecology for ultrasound scans where CO₂ is supplied into the abdominal cavity in order to separate the space between individual organs. Webpage available at the address https://www.alibaba.com/showroom/co2-carboxy-therapy-machine.html discloses a pen for cosmetic purposes that comprise CO₂ ampules. Such solutions do not deliver the needed amounts of CO₂ for successful treatments and the above described Bohr effect. Although subcutaneous CO₂ injections can deliver 100% CO₂, they are invasive, involve risks for infection and are only local, meaning that coverage of larger parts of human body is challenging.

On the other hand, Sakai et al (2011, PloS One, 6, 9: e24137) used a device for transcutaneous CO₂ delivery comprising a wrap for covering a part of the body and allowing supply of 100% CO₂ together with a hydrogel, so that the supplied CO₂ remained in the wrap. Similar approach was used by Ueha et al (2017; https://doi.org/10.3892/or.2017.5591), where transcutaneous administration of CO₂ to the area of skin around the tumour was achieved with a CO₂ hydrogel. The area was then sealed with a polyethylene bag, and 100% CO₂ gas was delivered into the bag.

The solution shown on the web page https://www.airjectorvet.com/ comprises a bag placed around the part of an animal that has to be treated with CO₂. The latter is introduced into the sealed bag with an especially adapted gun. After the treatment the CO₂ is released into the environment, which must be an open space to prevent intoxications with the gas. This solution differs from the present invention in many aspects, most importantly the gun does not allow introduction of concentrated CO₂ into the bag. Further, the present invention has a controlled release of CO₂ so that users of the device and the medical staff are safe.

### SUMMARY OF THE INVENTION

The invention is intended for performing transcutaneous CO₂ application treatments of all kinds of chronic or acute wounds and conditions where the basic problem is insufficient blood supply. Transcutaneous CO₂ administration is enabled by law of diffusion from the part with high CO₂ concentration (chamber of the device) to the part with a lower CO₂ concentration (the patient's body or body part). By introducing significant amounts of CO₂ onto the patient's body or body part blood circulation is improved, as well as nutritive perfusion of the treated area, which is crucial for faster healing of chronic wounds and neuropathy.

In a broad aspect of the invention, a device comprises at least:
a therapeutic chamber comprising at least a portion to receive a part of a patient's body to which the carbon dioxide is to be applied,
an inlet/outlet pipe connecting the chamber with a CO₂ distribution system, wherein the inlet/outlet pipe is connected to the chamber with a suitable element or a valve;
the CO₂ distribution system comprising a housing where at least the following components are installed:
   a first pipe with a first valve for suction of air out of the chamber,
   a second pipe with a second valve for supplying CO₂ from a tank/reservoir; wherein the first and second pipe are combined into the inlet/outlet pipe upstream of the valves;
   at least one device for ensuring air flow through the said valves and inlet/outlet pipe, preferably a ventilator or a pump;
   preferably at least one airflow measuring device for measuring the air flow through the valve on the inlet/outlet pipe or through any valve;
   preferably a reservoir for CO₂, where it is stored at the pressure of 1 to 5 bar, connected to the second pipe;
   an outlet pipe for leading the used air from the chamber through the wall to the external environment of the building where the device is installed; and
at least one tank for storing CO₂ suitably connected to the CO₂ distribution system or preferably to the reservoir.

### BRIEF DESCRIPTION OF THE DRAWINGS

FIG. 1 An embodiment of the device according to the invention

FIG. 2 Detailed view of the device shown in FIG. 1

FIG. 3A Valves and piping inside the CO₂ distribution system connected to the chamber with the inlet/outlet pipe with two pipes

FIG. 3B Valves and piping inside the CO2 distribution system connected to the chamber with the inlet/outlet pipe with three pipes

FIG. 4 The gate valve for supplying gas into the chamber from the inlet/outlet pipe

FIG. 5 Installation of the gate valve shown in FIG. 4 into the chamber

FIG. 6 Outlet pipe with the telescopic intra-wall part

FIG. 7 installation of telescopic intra-wall part in the wall of a building

### DESCRIPTION OF THE INVENTION

The invention is intended for performing transcutaneous CO₂ application treatments of all kinds of chronic or acute wounds and conditions where the basic problem is insufficient blood supply. Transcutaneous CO₂ administration is enabled by law of diffusion from the part with high CO₂ concentration (chamber of the device) to the part with a lower CO₂ concentration (the patient's body or body part). By introducing significant amounts of CO₂ onto the patient's body or body part blood circulation is improved, as well as nutritive perfusion of the treated area, which is crucial for faster healing of chronic wounds and neuropathy.

The essence of the device for transcutaneous application of carbon dioxide for treatment of chronic wounds or neuropathy is in that the device comprises at least:
a therapeutic chamber comprising at least a portion to receive a part of a patient's body to which the carbon dioxide is to be applied,
an inlet/outlet pipe connecting the chamber with a CO₂ distribution system, wherein the inlet/outlet pipe is connected to the chamber with a suitable element or a valve;
the CO₂ distribution system comprising a housing where at least the following components are installed:
   a first pipe with a first valve for suction of air out of the chamber,
   a second pipe with a second valve for supplying CO₂ from a tank/reservoir; wherein the first and second pipe are combined into the inlet/outlet pipe upstream of the valves;
   at least one device for ensuring air flow through the said valves and inlet/outlet pipe, preferably a ventilator or a pump;
   preferably at least one airflow measuring device for measuring the air flow through the valve on the inlet/outlet pipe or through any valve;
   preferably a reservoir for CO₂, where it is stored at the pressure of 1 to 5 bar, connected to the second pipe;
   an outlet pipe for leading the used air from the chamber through the wall to the external environment of the building where the device is installed; and
at least one tank for storing CO₂ suitably connected to the CO₂ distribution system or preferably to the reservoir.

The outlet pipe is connected to the inlet/outlet pipe through the first pipe of the CO₂ distribution system.

The device can be further provided with a filter in the CO₂ distribution system to filter out any impurities and/or a silencer for decreasing the sound resulting from the flow of gas under pressure. Said filter is preferably installed in the pipe of the CO₂ distribution system between the ventilator and the inlet/outlet pipe. It is attached in any suitable way, usually with clamps. The silencer is preferably installed between the second valve and the ventilator. The CO₂ distributing system may have more pipes and valves, preferably one additional pipe with a valve is provided for delivering air to the chamber before it is filled with CO₂ in order to achieve CO₂ concentrations below 100%.

The device may be further equipped with suitable electronics for easier controlling and managing of the device, wherein the controller has suitable buttons connected with a control device that opens and closes the valves used in the CO₂ distribution system, so that any particular CO₂ concentration in the chamber may be achieved.

The chamber comprising at least a portion to receive a part of a patient's body to which the carbon dioxide is to be applied can be a flexible (soft) chamber such as a wrap or it can be designed as a chamber with supporting elements enabling a certain geometry of the chamber when it is filled with air and/or carbon dioxide. The chamber may have different sizes, wherein it can be appropriately small to receive only a foot, a part of a leg, a whole leg, an arm or a part of an arm or it can be bigger to accommodate the whole body of a patient with the exception of patient's head. Possible materials for the chamber are all biocompatible materials, which are not permeable to CO₂, wherein the preferred choice is polyethylene, especially low-density polyethylene. The chamber is preferably for single use for hygienic reasons to prevent possible transfer of infections.

The inlet/outlet pipe to the chamber is only one, thereby rendering the construction of the device simpler. Further, its maintenance is easier. The inlet/outlet pipe is coupled to the chamber with a suitable passage valve (the gate valve), preferably the valve is comprising a rotating part and a static part, the latter being adapted for introduction into the inlet/outlet pipe. The rotating part has two removable parts, which are screwed into place from the interior of the chamber. Thereby the passage valve allows safe supply and suction of air to and from the chamber. When the chamber is flexible (soft) as a wrap, the gate valve is necessary.

The device can be preferably equipped with the reservoir for storing CO₂ at pressure from 1 to 5 bar. The reservoir is connected to the gas tank, where the gas is stored at pressures around 50 bars. Presence of the reservoir allows faster filling of the chamber, as the gas is already in gaseous state, at a suitable temperature and at suitable pressure. Namely, a part of the gas is led from the gas tank to the reservoir, where it due to a larger space expands, thereby also warming without any heaters.

Airtightness of the CO₂ distribution system inside the housing is ensured by using suitable seals and/or welding all metal components to each other, meaning that the valves are welded to the pipes, while the pipes are welded to the housing of the ventilator. The inlet/outlet pipe is connected to the system with a clamp and all attachments are provided with suitable seals, so that the CO₂ does not leak into the room where the device is used. The ventilator is preferably housed in a two-part housing welded together, wherein the housing is provided with a suitable number of holes for attachment of the required number of pipes. The valves may be any suitable, including electromagnetic or mechanic, controlled in any suitable way. The pipes are preferably metal, but can also be made of plastic materials, wherein welding is not possible but can be replaced with suitable seals such as rubber, silicon, Teflon and similar seals known in the art.

The outlet pipe for leading the used air from the chamber through the wall to the external environment of the building where the device is installed is preferably equipped with a telescopic transition for easy adjustment to a wide variety of walls, which often have different thicknesses. The telescopic transition may be extended or retracted depending on the wall, wherein its construction enables that it discharges the whole air from the chamber to the environment. The telescopic transition also seals the passage through the wall and prevents escape or return of the gas back into the room with the device according to the invention. The inner part of the telescopic transition may be provided with a seal, preferably installed on the flange of the inner part.

The first preferred embodiment of the device for transcutaneous application of carbon dioxide for treatment of chronic wounds or neuropathy is in that the device comprises:
a therapeutic chamber comprising at least a portion to receive a part of a patient's body to which the carbon dioxide is to be applied,
an inlet/outlet pipe connecting the chamber with a CO2 distribution system;
the CO₂ distribution system comprising a housing where at least the following components are installed:
   a first pipe with a first valve for suction of air out of the chamber, and a first airflow measuring device for measuring the air flow through the first valve,
   a second pipe with a second valve for supplying air to the chamber and a second air flow measuring device for measuring the air flow through the second valve,
   a third pipe with a third valve for supplying CO₂ from a tank or a reservoir and a third air flow measuring device for measuring the air flow through the third valve;
wherein the first, second and third pipe are combined into the inlet/outlet pipe upstream of the valves;
   one ventilator, preferably two, for ensuring air flow through the said valves and inlet/outlet pipe,
   preferably a reservoir for CO₂, where the gas is stored at the pressure of 1 to 5 bar, connected to the third pipe;
   an outlet pipe for leading the used air from the chamber through the wall to the external environment of the building where the device is installed; and
at least one tank for storing CO₂ suitably connected to CO₂ distribution system or preferably to the reservoir.

Said airflow measuring devices on individual pipes and valves may be replaced with one airflow measuring device installed on the inlet/outlet pipe and preferably also measuring the airflow through the gate valve. Every embodiment may be further equipped with a silencer and/or a filter and/or electronics for control. In case two ventilators are used, they are rotating in different directions with regards to the airflow (into the chamber or out of the chamber).

The second preferred embodiment of the device for transcutaneous application of carbon dioxide for treatment of chronic wounds or neuropathy is in that the device comprises:
a therapeutic chamber comprising at least a portion to receive a part of a patient's body to which the carbon dioxide is to be applied,
an inlet/outlet pipe connecting the chamber with a CO₂ distribution system,
the CO₂ distribution system comprising a housing where at least the following components are installed:
   a first pipe with a first valve for suction of air out of the chamber,
   a second pipe with a second valve for supplying CO₂ from a tank or reservoir;
   wherein the first and second pipe are combined into the inlet/outlet pipe upstream of the valves;
   a ventilator in a sealed housing for ensuring air flow through the said valves and inlet/outlet pipe,
   preferably a reservoir for CO₂, where the gas is stored at the pressure of 1 to 5 bar, connected to the second pipe;
   preferably a silencer for decreasing the sound resulting from the flow of gas under pressure;
   preferably a removable filter for filtering any impurities so that they do not reach the chamber;
   an outlet pipe for leading the used air from the chamber through the wall to the external environment of the building where the device is installed; and
at least one tank for storing CO₂ suitably connected to the CO₂ distribution system or preferably to the reservoir.

Presence of at least two valves and suitable piping ensures that the carbon dioxide concentration is adapted to the needs of therapy.

The three valves (first, second, third) and at least one air flow measuring device in the first preferred embodiment enable controlling air composition inside the chamber, so that different CO₂ concentrations can be achieved. The preferred range of CO₂ concentration inside the chamber is 10 to 100%, wherein most preferred range is between 30 and 90%. For example, if a 30% (V/V) concentration is needed inside the chamber and the total volume of the chamber is 100 L, 70 L of air will be supplied through the second valve and 30 L of CO₂ will be supplied through the third valve. A 90% (V/V) concentration may be achieved by supplying 10 L of air through the second valve and 90 L of CO₂ through the third valve.

Preferably, the valves are controlled with a controller or a suitable computer/computer program in order to ensure correct CO₂ concentrations inside the chamber. The controller preferably has a button for emptying the chamber, which can turn on ventilator and open the first valve for air suction. All other valves are closed. When the chamber is completely empty, the ventilator is turned off and the first valve is closed. Then the actual volume of the chamber is determined by filling it with air, wherein information is obtained by measuring air flow. Based on the actual volume of the chamber the amount of air has to be pumped out and which amount of CO₂ has to be led into the chamber. When the chamber is full the operator stops filling by pressing a suitable button. After the therapy is done, a discharge button provided on the controller is pushed and this leads to the first valve being open and the second valve is closed, so that all air/CO₂ from the chamber is led to the exterior of the building.

The two valves and the airflow measuring device in the second preferred embodiment enable controlling of CO₂ concentrations inside the chamber so that the air is sucked from the chamber through the first pipe with the first valve and CO₂ is supplied from the tank or the reservoir to the chamber through the second pipe with the second valve. Thereby a CO₂ concentration near 100% is achieved in the chamber. If a lower concentration of CO₂ is required, the chamber is initially not emptied, but a certain amount of air may be left inside it. This embodiment has simpler operation and requires no electronics for control, although they are preferred.

The functioning method of the said device comprises the following steps:
in case of flexible chamber, first attaching the chamber to the inlet-outlet pipe via the valve or the gate valve;
placing at least a part of patient's body into the chamber and sealing the chamber;
sucking out all air from the chamber with the first valve with a ventilator and leading all sucked air to the exterior of the building with the room where the device is used;
leading CO₂ from the tank to the CO₂ distribution system or preferably the reservoir for allowing the CO₂ to expand in the reservoir, which results in decrease of its pressure in an increase of its temperature;
closing the first valve and opening the second valve to supply a desired amount of air into the chamber and/or opening the third valve to supply a desired amount of 100% CO₂ into the chamber.

When the CO₂ administration is finished, the air with the CO₂ is sucked from the chamber through the outlet pipe and led outside of the building where the device is installed. When all air has been emptied from the chamber, it can be unsealed and removed, so that the patient may leave. Usually the therapy lasts for 10 minutes to up to 2 hours, wherein the length of each therapy can be adjusted based on the patient's state.

Use of the device is suitable for all patients with impaired microcirculation; especially but not limited to patients affected with: chronic and acute wounds, neuropathy, muscle tears, etc.

The invention has been tested on 47 patients (38 males and 9 females aged 65.4±12.0 years) with the total number of 61 diabetic wounds. Control group consisted of 26 patients (21 males and 5 females aged 66.5±10.7 years) with the total number of 31 diabetic wounds (median volume: 528 mm³, median area: 253 mm²), who underwent placebo treatment with transcutaneous application of air. Experimental group consisted of 21 patients (21 males and 5 females aged 66.5±10.7) with the total number of 30 diabetic wounds (median volume: 351 mm³, median area: 241 mm²), who underwent treatment with transcutaneous application of CO₂ using the invention. Testing lasted for 4 weeks and laser Doppler blood perfusion in foot skin microcirculation, heart rate and blood pressure measurements were carried out. Further, each subject underwent monofilament and vibration sensation tests. After the treatments following main findings were observed:
1) 67% (20 out of 30) of all the wounds from the experimental groups were successfully healed, whereas the volume and area of the unhealed wounds on average decreased by 96% and 89%, respectively. None of the wounds from the control group was healed.
2) The results of the monofilament and vibration tests showed statistically significant improvement in terms of the sites with perceived monofilament/vibration stimulus for the experimental group.
3) Results of laser Doppler blood perfusion indicate that the function of the endothelial and neurogenic vascular tone regulating mechanisms of microcirculation improved significantly for the experimental group.
4) Absolute values of heart rate and arterial blood pressure before and after CO₂ therapies indicate that no systemic effects were caused during the treatment.

The device for transcutaneous application of carbon dioxide for treatment of chronic wounds or neuropathy, will be described in further detail based on possible embodiments and figures, which show:

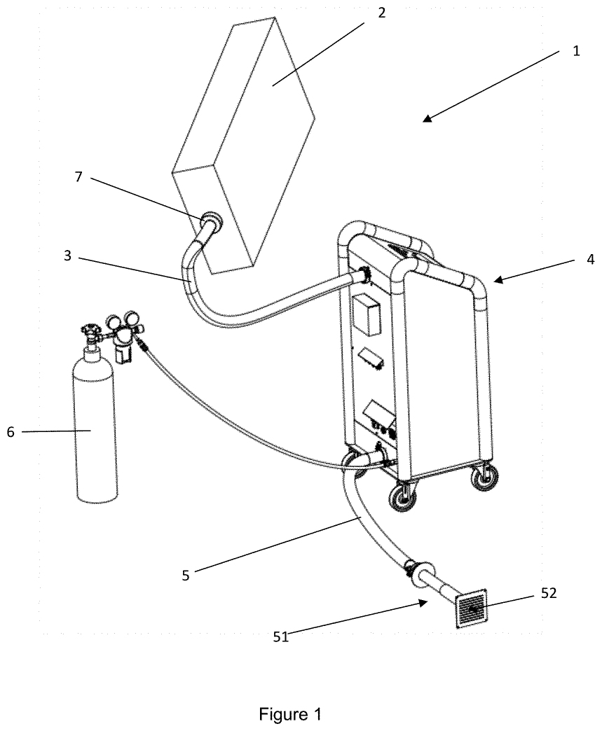

FIG. 1 shows an embodiment of the device 1 according to the invention, the device 1 comprising the following:
a therapeutic chamber 2 comprising at least a portion to receive a part of a patient's body to which the carbon dioxide is to be applied,
one inlet-outlet pipe 3 connecting the chamber 2 with a CO₂ distribution system 4, wherein a gate valve 7 is preferably used, especially if the chamber 2 is flexible;
the CO₂ distribution system 4;
an outlet pipe 5 for leading the used air from the chamber 2 through a telescopic part 51 and an outlet 52 in a wall to the external environment of the building where the device is installed; and
at least one tank 6 for storing CO₂ suitably connected to the CO₂ distribution system.

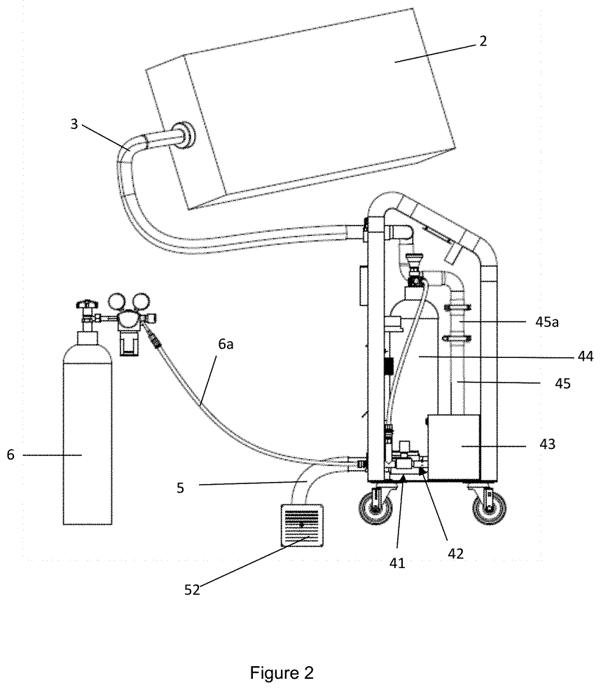

FIG. 2 shows a detailed view of the preferred embodiment of the device, wherein CO₂ is provided from the tank into a reservoir 44 forming a part of the CO₂ distribution system. The CO₂ expands in the reservoir and can be then supplied to the chamber via the piping, valves and ventilator as described above. As said above, this enables that the CO₂ is at pressure 1 to 5 bar, which speeds up filling of the chamber. The CO₂ distribution system 4 has at least a first pipe 41 and a second pipe 42, connected to a ventilator 43, which is connected to the inlet/outlet pipe 3 with a pipe 45, which is preferably equipped with a filter 45a. When the CO₂ administration is finished, the CO₂ is led out through the first pipe 41 to the outlet pipe 5 into the exterior of the building.

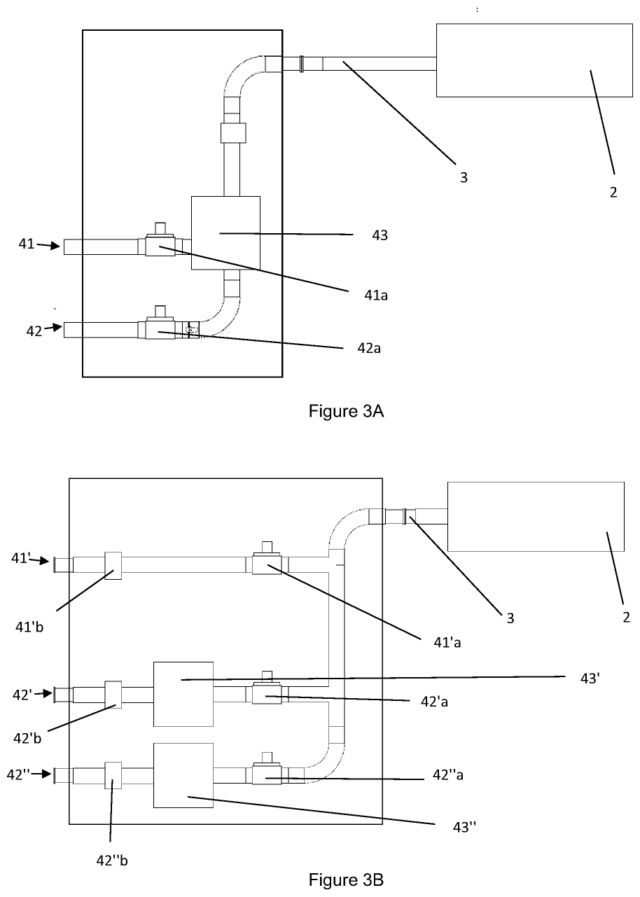

FIG. 3a shows a possible embodiment of the CO₂ distribution system comprising a housing where two pipes 41,42 are installed, each pipe having its own valve 41a, 42a, and both pipes connected to the ventilator 43, which is then connected to the inlet/outlet pipe 3 and consequently the chamber 2.

FIG. 3b shows the preferred embodiment of the CO₂ distribution system comprising a housing where the following components are installed:
a first pipe 41′ with a first valve 41′a for supplying CO₂ from the reservoir or the tank;
a second pipe 42′ with a second valve 42′a for supplying air from the environment;
a third pipe 42″ with a third valve 42″a for suction of air out of the chamber; wherein the said pipes are combined into the inlet/outlet pipe 3 upstream of the valves;
two ventilators 43′ and 43″ for ensuring air flow through the said valves and inlet/outlet pipe,
at least one airflow measuring device 41′b, 42′b, 42″b for measuring the air flow through the gate valve on the inlet/outlet pipe or through any valve;
the reservoir for CO₂, where it is stored at the pressure of 1 to 5 bar, connected to the third pipe;

Pipes, valves, ventilator and the reservoir are connected airtightly with suitable welding and sealing. The valves may be any suitable valves such as electromagnetic or mechanical valves, wherein the gate valve has to be present in case the chamber is designed as a flexible wrap.

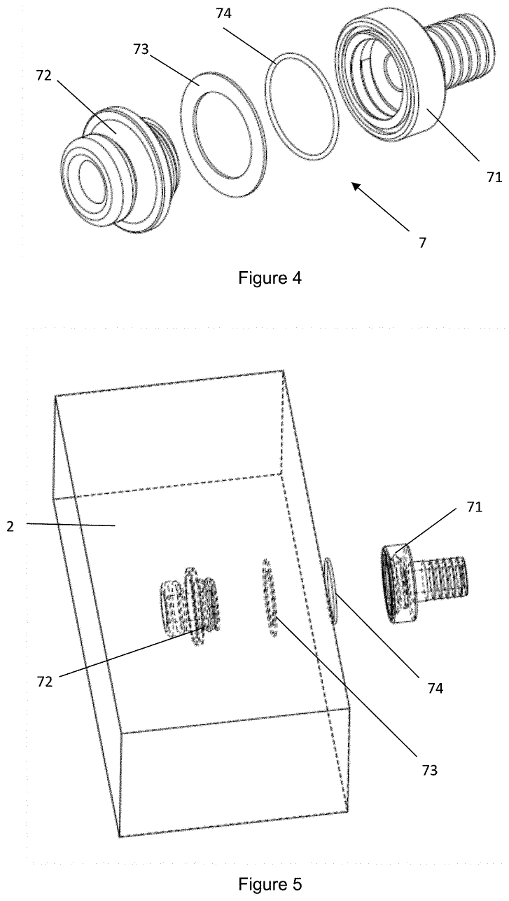

The gate valve 7 for connecting the inlet/outlet pipe 3 to the chamber 2 is shown in FIG. 4 and it comprises:
a rotating part 72;
preferably a washer 73;
a seal 74; and
a static part 71, the latter being adapted for introduction into the inlet/outlet pipe;

wherein the rotating part has two removable parts, which are screwed into place from the interior of the chamber. The seal 74 is installed in the static part, while the washer prevents rotation of the chamber, when the rotating part is screwed into place with the static part 71. The washer is especially useful for flexible chambers, while it is not needed in hard chambers. Preferably the gate valve is made of biocompatible medical grade plastics. FIG. 5 shows installation of the gate valve 7, wherein the rotating part 72 and the washer 73 are installed from the interior of the chamber, while the seal and the static part are installed from the direction of the inlet/outlet pipe 3. The rotating part 72 and the static part 71 have corresponding treads, which can interlock so that air leakage is prevented. This is further ensured with the seal 74.

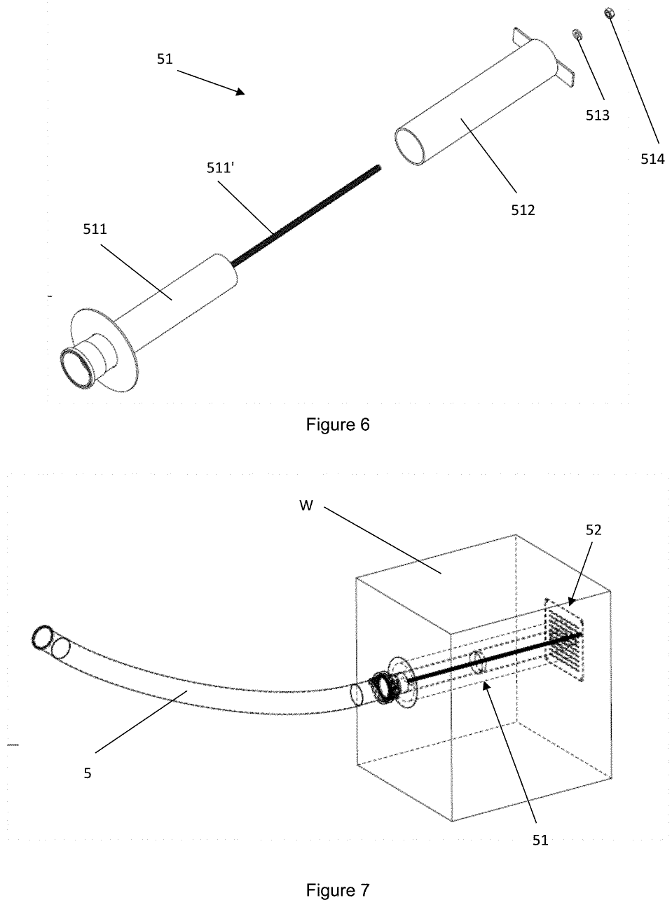

FIGS. 6 and 7 show the telescopic intra-wall part 51 and its installation into the wall W. The telescopic part 51 comprises:
an inner part 511; the inner part's flange optionally provided with a seal for improved sealing
an outer part 512;
a washer 513; and
a nut 514.

The wall has to be equipped with a pre-prepared hole having a diameter between 10 mm to 100 m, into which the telescopic part 51 is installed. The smaller inner part 511 can move along the outer part 512, wherein a threaded pole 511′ runs through the entire length of the telescopic part 51. A second nut is provided for tightening. The telescopic part can be adjusted so that it corresponds to the thickness of the wall W into which it is to be installed. The outlet 52 can be performed in any suitable way. Air can travel through the telescopic part into the interior of the CO₂ distribution system, more precisely towards the first pipe 41, or can travel to the exterior of the building when the therapy is over.

Within the scope of the invention as described herein and defined in the claims, other embodiments of the device for transcutaneous application of carbon dioxide for treatment of chronic wounds or neuropathy that are clear to person skilled in the art may be possible, which does not limit the essence of the invention as described herein and defined in the claims.

1. A device for transcutaneous application of carbon dioxide for treatment of chronic wounds or neuropathy comprising at least:
a therapeutic chamber comprising at least a portion to receive a part of a patient's body to which the carbon dioxide is to be applied,
an inlet/outlet pipe connecting the chamber with a CO₂ distribution system, wherein the inlet/outlet pipe is connected to the chamber with a suitable element or a valve;
the CO₂ distribution system comprising a housing where at least the following components are installed:
a first pipe with a first valve for supplying CO₂ from a tank or a reservoir,
a second pipe with a second valve for suction of air out of the chamber;
wherein the first and second pipe are combined into the inlet/outlet pipe upstream of the valves;
at least one device for ensuring air flow through the said valves and inlet/outlet pipe, preferably a ventilator or a pump;
preferably at least one airflow measuring device for measuring the air flow through the valve on the inlet/outlet pipe or through any valve;
preferably a reservoir for CO₂, where it is stored at the pressure of 1 to 5 bar, connected to the second pipe;
an outlet pipe for leading the used air from the chamber through the wall to the external environment of the building where the device is installed; and
at least one tank for storing CO₂ suitably connected to the CO₂ distribution system or preferably to the reservoir.

2. The device according to claim 1, characterized in that characterized in that the CO₂ distributing system may have more pipes and valves, preferably one additional pipe with a valve is provided for delivering air to the chamber before it is filled with CO₂ in order to achieve CO₂ concentrations below 100%.

3. The device according to claim 1, characterized in that the device may have one airflow measuring device for measuring airflow through the inlet/outlet pipe or several airflow measuring devices installed on each pipe of the CO₂ distribution system for measuring airflow through the valve on each pipe.

4. The device according to claim 1, characterized in that the inlet/outlet pipe is coupled to the chamber with a gate valve, preferably the valve comprises:
a rotating part;
preferably a washer;
a seal; and
a static part adapted for introduction into the inlet/outlet pipe;
wherein the rotating part has two removable parts, which are screwed into place from the interior of the chamber, the seal is installed in the static part, while the preferred washer prevents rotation of the chamber, when the rotating part is screwed into place with the static part.

5. The device according to claim 1, characterized in that the chamber is for single use.

6. The device according to claim 1, characterized in that the chamber is a flexible chamber such as a wrap or is designed as a chamber with supporting elements enabling certain geometry of the chamber when it is filled with air and/or CO₂, wherein the chamber may have any size and made of any biocompatible material not permeable to CO₂, the preferred choice is polyethylene, especially low-density polyethylene; provided that the chamber designed as a wrap has the gate valve according to claim 4 for connection with the inlet/outlet pipe.

7. The device according to claim 1, characterized in that the device is provided with the reservoir for storing CO₂ at pressure from 1 to 5 bar, the reservoir being connected to the gas tank, where the gas is stored at pressures around 50 bars.

8. The device according to claim 1, characterized in that the CO₂ distribution system inside the housing is airtight, which is ensured by suitable seals for non-metal components and/or welding all metal components to each other, meaning that the valves are welded to the pipes, while the pipes are welded to the housing of the ventilator; and that the inlet/outlet pipe is connected to the system with a clamp and all attachments are provided with suitable seals, so that the CO₂ does not leak into the room where the device is used.

9. The device according to claim 1, characterized in that the valves may be any suitable, including electromagnetic or mechanic, controlled in any suitable way.

10. The device according to claim 1, characterized in that the outlet pipe for leading the used air from the chamber through the wall to the external environment of the building where the device is installed is equipped with a telescopic transition for easy adjustment to a wide variety of walls, wherein the telescopic part comprises:
an inner part, the inner part's flange optionally provided with a seal for improved sealing;
an outer part;
a washer; and
a nut for tightening;
wherein the smaller inner part can move along the outer part and a threaded pole runs through the entire length of the telescopic part.

11. The device according to claim 1, characterized in that it is further provided with a filter in the CO₂ distribution system to filter out any impurities and/or a silencer for decreasing the sound resulting from the flow of gas under pressure, wherein said filter is preferably installed in the pipe of the CO₂ distribution system between the ventilator and the inlet/outlet pipe and the silencer is preferably installed between the second valve and the ventilator.

12. The device according to claim 1, characterized in that it is further equipped with suitable electronics for easier controlling and managing of the device, wherein the controller has suitable buttons connected with a control device that opens and closes the valves used in the CO₂ distribution system, so that any particular CO₂ concentration in the chamber may be achieved.

13. The device according to claim 1 for use in therapy, especially in therapy of patients with impaired microcirculation; especially but not limited to patients affected with: chronic and acute wounds, neuropathy, muscle tears, etc.

14. The device according to claim 13, characterized in that the preferred range of CO₂ concentration inside the chamber is 10 to 100%.

15. A functioning method of the device according to claim 1, the method comprises the following steps:
in case of flexible chambers, first attaching the chamber to the inlet-outlet pipe via the valve or the gate valve;
placing at least a part of patient's body into the chamber and sealing the chamber;
sucking out all air from the chamber with the first valve with a ventilator and leading all sucked air to the exterior of the building with the room where the device is used;
leading CO₂ from the tank to the CO₂ distribution system or preferably the reservoir for allowing the CO₂ to expand in the reservoir, which results in decrease of its pressure in an increase of its temperature;
closing the first valve and opening the second valve to supply a desired amount of air into the chamber and/or opening the third valve to supply a desired amount of 100% CO₂ into the chamber; and
when the CO₂ administration is finished, the air with the CO₂ is sucked from the chamber through the outlet pipe and led outside of the building where the device is installed.
